(19) United States
(12) Patent Application Publication
(10) Pub. No.: US 2006/0024844 A1
(43) Pub. Date: Feb. 2, 2006

(54) Method for the idenification of ligands

(76) Inventors: Dionisios Rentzeperis, Downingtown, PA (US); Hossein Askari, Exton HA., PA (US); Barry A. Springer, Wilmington, DE (US); Roger F. Bone, Bridgewater, NJ (US); Francis R. Salemme, Yardley, PA (US)

Correspondence Address: WILSON SONSINI GOODRICH & ROSATI, 650 PAGE MILL ROAD, PALO ALTO, CA, 94304-1050, US

(21) Appl. No.: 10/522,660

(22) PCT Filed: Sep. 20, 2002
(86) PCT No.: PCT/US02/29661

§ 371 Date: Aug. 16, 2005

Related U.S. Application Data
(60) Provisional application No. 60/398,023, filed on Jul. 24, 2002.

(51) Int. Cl. G01N 33/543; G06F 19/00; G01N 33/48; G01N 33/50

(52) U.S. Cl. 436/518; 702/19

(57) ABSTRACT

The present invention relates generally to a method of identifying ligands that modulate protein-protein interactions. More particularly, the present invention relates to methods of determining agonists or antagonists of a co-regulator dependent target molecule based on the ability to modify the stability of the target molecule.

This application claims priority benefit of U.S. Provisional Application No. 60/398,023 filed Jul. 24, 2002, which is incorporated by reference herein in its entirety.

### FIELD OF THE INVENTION

The present invention relates generally to a method of identifying ligands for protein-protein interactions whose affinity is modulated by ligands or allosteric regulators. More particularly, the present invention relates to methods of determining agonist or antagonist ligands of a receptor based on the ability to modify the stability of the receptor when in the presence of a co-regulator.

### BACKGROUND OF THE INVENTION

A central theme in signal transduction and gene expression is the constitutive or inducible interaction of protein-protein modular domains. Knowledge of ligands that can potentiate these interactions will provide information on the nature of the molecular mechanisms underlying biological events and on the development of therapeutic approaches for the treatment of disease. Existing methods for the identification of ligands are cumbersome and limited particularly in the case of proteins of unknown function.

Nuclear receptors are members of a superfamily of transcription factors controlling cellular functions including reproduction, growth differentiation, lipid and sugar homeostasis. Their function is regulated by a diverse set of ligands (xenobiotics, hormones, lipids and other known and undiscovered ligands). To date 48 nuclear receptors have been identified, 28 with known ligands and the remaining ones are classified as orphans. The biology of the receptors is complex and tissue specific (Shang & Brown, Science 295:2465-2468, 2002) and the molecular mechanism of action appears to be a function of preferential recruitment of accessory proteins, referred as co-regulators, that modulate the function of these receptors in a ligand independent or dependent fashion. Recruitment of the appropriate co-regulator can result in gene transcription or repression.

Panvera offers reagents for the discrimination of agonist from antagonist ligands for the estrogen receptor subtype beta and has presented publicly data on the preferential recruitment of co-activator proteins (Bolger et al., Environmental Health Perspectives 106:1-7 (1998); Panvera corporate presentation presented at the Orphan Receptor Meeting San Diego, June 2002). Their reagents are used in assays based on fluorescence resonance energy transfer (FRET).

There are publications on similar assays for other nuclear receptors (ER-α, the ERR and PPAR family) that are also based on FRET (Zhou et al., Molecular Endocrinology 12:1594-1604 1998)), (Coward et al., 98:8880-8884, (2001)). Similar experiments have been done using Biacore technology (Cheskis et al., J. Biological Chemistry 11384-11391 (1997))(Wong et al.; Biochemistry 40:6756-6765 (2001)).

Cellular assays exist where the readout is gene expression (Camp et al., Diabetes 49:539-547 (2001))(Kraichely et al., Endocrinology, 141:3534-3545, (2000)). For example, Karo-Bio has developed a gene expression readout assay to include conformational sensitive peptide probes for discrimination of agonist from antagonist ligands for nuclear hormone receptors (Paige et al., PNAS 96:3999-4004 (1999)), (presentation by Karo-Bio at the Orphan Receptor Meeting, San Diego June 2002).

Greenfield et al., Biochemistry 40:6446-6652 (2001) reports the thermal stablization of the ER-α receptor in the presence of estradiol. However, the reference does not teach the identification of a molecule as an agonist or an antagonist of the ER-α receptor.

The art discussed above suffers from several drawbacks. For example, in the analysis of nuclear receptors, gene expression readout assays and cell based assays, counter-screens are required to validate that ligands or co-regulators identified interact directly with the receptor of interest and not through other proteins that can produce a signal transduction or gene activation/repression assay readout. In addition, cell readout technology lacks the sensitivity in identifying weak ligands (typically compounds of affinities of greater than 1 μM are rarely identified), and is only applicable to compounds that have a good cell permeability profile. Other commercial in vitro assays require the knowledge of ligands for establishing competitive displacement assays, or the use of them as tools to validate FRET based co-regulator assays.

Thus, there is a need for an accurate, reliable technology that facilitates the rapid, high-throughput identification of ligands for co-regulator-dependent receptors and further identification of their effect on the receptor when in the presence of a co-regulator.

### SUMMARY OF THE INVENTION

The present invention meets such needs. The present invention provides a method of identifying an agonist or an antagonist of a co-regulator-dependent target molecule. The method comprises providing a set of molecules that modify the stability of the target molecule and screening one or more molecules of the set for their ability to further modify the stability of the target molecule in the presence of one or more co-regulators. A further modification of the stability of the target molecule in the presence of a molecule of the set and a co-regulator indicates whether the molecule is an agonist or an antagonist of the target molecule when in the presence of the co-regulator.

The invention provides another method of identifying an agonist or an antagonist of a co-regulator-dependent target molecule. The method comprises providing a set of molecules that shift the thermal unfolding curve of the target molecule and screening one or more of the molecules of the set for their ability to further shift the thermal unfolding curve of the target molecule in the presence of one or more co-regulators. A further shift in the thermal unfolding curve of the target molecule in the presence of a molecule of the set and a co-regulator indicates whether the molecule is an agonist or an antagonist of the target molecule when in the presence of the co-regulator.

The present invention also provides a method of identifying an antagonist of a co-regulator-dependent target molecule. The method comprises providing a set of molecules that modify the stability of the target molecule and screening one or more molecules of the set for their ability to further modify the stability of the target molecule in the presence of one or more co-activators. If there is no further modification of the stability of the target molecule in the presence of a molecule of the set and a co-activator, this is an indication that the molecule of the set is an antagonist of the target molecule when in the presence of the co-activator.

The present invention provides another method of identifying an antagonist of a co-regulator-dependent target molecule. The method comprises providing a set of molecules that shift the thermal unfolding curve of the target molecule and screening one or more of the molecules of the set for their ability to further shift the thermal unfolding curve of the target molecule in the presence of one or more co-activators. If there is no further shift in the thermal unfolding curve in the presence of a molecule of the set and a co-activator, this is an indication that the molecule of the set is an antagonist of the target molecule when in the presence of the co-activator.

The present invention also provides a method of identifying an agonist of a co-regulator-dependent target molecule. The method comprises providing a set of molecules that modify the stability of the target molecule and screening one or more molecules of the set for their ability to further modify the stability of the target molecule in the presence of one or more co-repressors. If there is no further modification of the stability of the target molecule in the presence of a molecule of the set and a co-repressor, this is an indication that the molecule of the set is an agonist of the target molecule when in the presence of the co-repressor.

The present invention provides another method of identifying an agonist of a co-regulator-dependent target molecule. The method comprises providing a set of molecules that shift the thermal unfolding curve of the target molecule and screening one or more of the molecules of the set for their ability to further shift the thermal unfolding curve of the target molecule in the presence of one or more co-repressors. If there is no further shift in the thermal unfolding curve in the presence of a molecule of the set and a co-repressor, this is an indication that the molecule of the set is an agonist of the target molecule when in the presence of the co-repressor.

The present invention also provides a method for determining an agonist or an antagonist of a target molecule having an unknown function. The method comprises providing a set of molecules that modify the stability of a target molecule having an unknown function, wherein the set of molecules modify the stability of receptors which share biological function, and screening one or more molecules of the set for their ability to further modify the stability of the target molecule in the presence of one or more co-regulators. A further modification of the stability of the target molecule in the presence of a molecule of the set and a co-regulator indicates whether the molecule is an agonist or an antagonist of the target molecule when in the presence of the co-regulator.

The present invention provides another method for determining an agonist or an antagonist of a target molecule having an unknown function. The method comprises providing a set of molecules that shift the thermal unfolding curve of a target molecule having an unknown function, wherein the set of molecules shift the thermal unfolding curve of receptors which share biological function, and screening one or more molecules of the set for their ability to further shift the thermal unfolding curve of the target molecule in the presence of one or more co-regulators. A further shift in the thermal unfolding curve of the target molecule in the presence of a molecule of the set and a co-regulator indicates whether the molecule is an agonist or an antagonist of the target molecule when in the presence of the co-regulator.

An advantage of the methods of the present invention is that neither gene expression readout and cell based assays, nor the use of known ligands to establish the assay are required. The ability to generate information in such a direct fashion allows the discovery of drugs with desired properties, to test therapeutic hypotheses and decrypt orphan receptors.

By use of isolated and/or purified proteins and peptides in a single unifying assay, one can identify ligands that are involved in modulating protein-protein interactions and predict biological response. Not only can ligands be identified, but also the intrinsic affinity for the target protein can be calculated which then can be used to correlate to biological activity. The information generated can also be used to identify ligands for orphan receptors that in turn can be used as tools to deconvolute the biology of these proteins to test therapeutic hypotheses.

Data generated by methods of the present invention does not require counter-screening, as changes in the melting temperature of a target molecule, such as a protein is a direct consequence of the thermodynamic linkage of the binding energy of macromolecules and ligands to the protein of interest. Further, affinities of a ligand to a target molecule are more sensitive (affinities of pM to mM are determined). Further, the present invention is not limited by compounds with poor cell permeability. Also, as mentioned above, the present invention does not require known ligands to establish an assay, making it extremely powerful for deconvoluting orphan receptors.

Further features and advantages of the present invention are described in detail below with reference to the accompanying drawings.

### BRIEF DESCRIPTION OF THE FIGURES

FIG. 1 illustrates experimental results expected for the identification of an agonist ligand in the presence of a co-activator.

FIG. 2 illustrates experimental results expected for the identification of an antagonist ligand in the presence of a co-activator.

### DETAILED DESCRIPTION

In the following description, reference will be made to various terms and methodologies known to those of skill in the biochemical and pharmacological arts. Publications and other materials setting forth such known terms and methodologies are incorporated herein by reference in their entireties as though set forth in full.

In embodiments of the present invention, methods are provided for the identification of agonists and antagonists for co-regulator-dependent target molecules, which are capable of unfolding, based upon molecules that modify the stability of the target molecule. Molecules that modify the stability of the target molecule can be screened in the presence of the target molecule and one or more co-regulators for their ability to further modify the stability of the target molecule. Whether the stability of the target molecule is further modified is an indication as to whether the molecule is an agonist or an antagonist of the target molecule when in the presence of the co-regulator.

In other embodiments of the invention, methods are provided for the identification of agonists and antagonists for co-regulator-dependent target molecules which involve the unfolding of a target molecule due to a thermal change. Molecules that shift the thermal unfolding curve of the target molecule can be screened in the presence of the target molecule and one or more co-regulators for their ability to further shift the thermal unfolding curve of the target molecule. Whether the thermal unfolding curve of the target molecule is further shifted is an indication as to whether the molecule is an agonist or an antagonist of the target molecule when in the presence of the co-regulator.

The term “target molecule” encompasses peptides, proteins, nucleic acids, and other receptors. The term encompasses both enzymes and proteins which are not enzymes. The term encompasses monomeric and multimeric proteins. Multimeric proteins may be homomeric or heteromeric. The term encompasses nucleic acids comprising at least two nucleotides, such as oligonucleotides. Nucleic acids can be single-stranded, double-stranded or triple-stranded. The term encompasses a nucleic acid which is a synthetic oligonucleotide, a portion of a recombinant DNA molecule, or a portion of chromosomal DNA.

The term “target molecule” also encompasses portions of peptides, proteins, and other receptors which are capable of acquiring secondary, tertiary, or quaternary structure through folding, coiling or twisting.

The target molecule may be substituted with substituents including, but not limited to, cofactors, coenzymes, prosthetic groups, lipids, oligosaccharides, or phosphate groups. The term “target molecule” and “receptor” are synonymous.

More specifically, the target molecules utilized in the present invention are co-regulator-dependent. By “co-regulator-dependent” it is meant that the target molecule is capable of binding at least one ligand and binding at least one co-regulator. Further, the activity of the target molecule, whether in a ligand dependent or independent function, is dependent upon, at least in part, by a co-regulator. Co-regulator dependent target molecules include, but are not limited to, nuclear receptors.

Nuclear receptors, and the role of co-regulators relating thereto, are described in Aranda and Pascual, Physiological Reviews 81:1269-1304 (2001); Collingwood et al., Journal of Molecular Endocrinology 23:255-275 (1999); Robyr et al., Molecular Endocrinology 23:329-347 (2000); and Lee et al., Cellular and Molecular Life Sciences 58:289-297 (2001), the references incorporated by reference herein by their entireties.

Further, the co-regulator dependent target molecules encompass vertebrate species, including, but not limited to humans, as well as invertebrates, including but not limited to insects.

Illustratively, insects contain hundreds of nuclear receptors, for which ligands can be identified as agonists or antagonists. See Laudet, J. Molecular Endocrinology 19:207-226 (1997) and Maglich et al., Genome Biology 2:1-7 (2001) for a discussion of nuclear receptors present in vertebrates, nematodes and arthropods, the references incorporated by reference herein by their entireties.

The term “protein” encompasses full length or polypeptide fragments. The term “peptide” refers to protein fragments, synthetic or those derived from peptide libraries. As used herein, the terms “protein” and “polypeptide” are synonymous.

The term “co-regulator” refers to chemical compounds of any structure, including, but not limited to nucleic acids, such as DNA and RNA, and peptides that modulate the target molecule in a ligand dependent or independent fashion. The term refers to natural, synthetic and virtual molecules. More specifically, the term refers to a peptide or polypeptide/protein, natural or synthetic that modulates the target molecule in a ligand dependent or independent fashion. The term encompasses peptides that are derived from natural sequences or from phage display libraries. The peptide can be fragments of native proteins. More specifically, the term refers to co-activators and co-repressors.

The term “co-activator” refers to a molecule which binds to a target molecule and causes an activation of or an increase in an activity of the target molecule. In embodiments of the invention, the term refers to molecules that bind to a target molecule to induce gene transcription or to induce a signaling function (e.g. signal transduction).

The term “co-repressor” refers to a molecule which binds to a target molecule and causes a deactivation or a decrease in an activity of the target molecule. In embodiments of the invention, the term refers to molecules that bind to a target molecule to repress gene transcription or to repress a signaling function (e.g. signal transduction).

The term “agonist” refers to a molecule which binds to a target molecule and induces or recruits a co-activator for binding to the target molecule.

In embodiments of the invention, the term “agonist” refers to a molecule that binds to a nuclear receptor and recruits a co-activator. In these embodiments, the term more specifically refers to a molecule that alters gene expression by inducing conformational changes in a nuclear receptor that promote direct interactions with co-activators.

The term “antagonist” refers to a molecule which binds to a target molecule and induces or recruits a co-repressor for binding to the target molecule.

In embodiments of the invention, the term “antagonist” refers to a molecule that binds to a nuclear receptor and recruits a co-repressor. In these embodiments, the term more specifically refers to a molecule that alters gene expression by inducing conformational changes in a nuclear receptor that promote direct interactions with co-repressors.

The term “molecule” refers to a compound which is tested for binding to the target molecule in the presence of or absence of additional compounds, such as co-regulators. This term encompasses chemical compounds of any structure, including, but not limited to nucleic acids, such as DNA and RNA, and peptides. The term refers to natural, synthetic and virtual molecules. The term includes compounds in a compound or a combinatorial library. The terms “molecule” and “ligand” are synonymous.

The terms “multiplicity of molecules,” “multiplicity of compounds,” or “multiplicity of containers” refer to at least two molecules, compounds, or containers.

The term “function” refers to the biological function of a target molecule, such as, e.g., a protein, peptide or polypeptide.

A “thermal unfolding curve” is a plot of the physical change associated with the unfolding of a protein or a nucleic acid as a function of temperature.

The terms “bind” and “binding” refer to an interaction between two or more molecules. More specifically, the terms refer to an interaction, such as noncovalent bonding, between a ligand and a target molecule, or a co-regulator and a target molecule, or a ligand, target molecule, and a co-regulator.

The term “modification of stability” refers to the change in the amount of pressure, the amount of heat, the concentration of detergent, or the concentration of denaturant that is required to cause a given degree of physical change in a target protein that is bound by one or more ligands, relative to the amount of pressure, the amount of heat, the concentration of detergent, or the concentration of denaturant that is required to cause the same degree of physical change in the target protein in the absence of any ligand. Modification of stability can be exhibited as an increase or a decrease in stability. Modification of the stability of a target molecule by a ligand indicates that the ligand binds to the target molecule.

The term “further modification of stability” refers to an additional modification of stability of the target molecule when in the presence of a molecule known to modify the stability of the target molecule and one or more additional molecules. More specifically, the one or more additional molecules can be co-regulators.

The term “unfolding” refers to the loss of structure, such as crystalline ordering of amino acid side-chains, secondary, tertiary, or quaternary protein structure. A target molecule, such as a protein, can be caused to unfold by treatment with a denaturing agent (such as urea, guanidinium hydrochloride, or guanidinium thiosuccicinate), a detergent, by treating the target molecule with pressure, by heating the target molecule, or by any other suitable change.

The term “physical change” encompasses the release of energy in the form of light or heat, the absorption of energy in the form or light or heat, changes in turbidity and changes in the polar properties of light. Specifically, the term refers to fluorescent emission, fluorescent energy transfer, absorption of ultraviolet or visible light, changes in the polarization properties of light, changes in the polarization properties of fluorescent emission, changes in the rate of change of fluorescence over time (i.e., fluorescence lifetime), changes in fluorescence anisotropy, changes in fluorescence resonance energy transfer, changes in turbidity, and changes in enzyme activity. Preferably, the term refers to fluorescence, and more preferably to fluorescence emission. Fluorescence emission can be intrinsic to a protein or can be due to a fluorescence reporter molecule. The use of fluorescence techniques to monitor protein unfolding is well known to those of ordinary skill in the art. For example, see Eftink, M. R., Biophysical J. 66: 482-501 (1994).

An “unfolding curve” is a plot of the physical change associated with the unfolding of a protein as a function of parameters such as temperature, denaturant concentration, and pressure.

The term “modification of thermal stability” refers to the change in the amount of thermal energy that is required to cause a given degree of physical change in a target protein that is bound by one or more ligands, relative to the amount of thermal energy that is required to cause the same degree of physical change in the target protein in the absence of any ligand. Modification of thermal stability can be exhibited as an increase or a decrease in thermal stability. Modification of the thermal stability of a target molecule by a ligand indicates that the ligand binds to the protein.

The term “shift in the thermal unfolding curve” refers to a shift in the thermal unfolding curve for a target molecule that is bound to a ligand, relative to the thermal unfolding curve of the protein in the absence of the ligand.

The term “further shift in the thermal unfolding curve” refers to an additional shift of the thermal unfolding curve of the target molecule when in the presence of a molecule known to shift the thermal unfolding curve of the target molecule and one or more additional molecules. More specifically, the one or more additional molecules can be co-regulators.

The term “contacting a target molecule” refers broadly to placing the target protein in solution with the molecule to be screened for binding. Less broadly, contacting refers to the turning, swirling, shaking or vibrating of a solution of the target molecule and the molecule to be screened for binding. More specifically, contacting refers to the mixing of the target molecule with the molecule to be tested for binding. Mixing can be accomplished, for example, by repeated uptake and discharge through a pipette tip. Preferably, contacting refers to the equilibration of binding between the target protein and the molecule to be tested for binding. Contacting can occur in the container or before the target molecule and the molecule to be screened are placed in the container.

The term “container” refers to any vessel or chamber in which the receptor and molecule to be tested for binding can be placed. The term “container” encompasses reaction tubes (e.g., test tubes, microtubes, vials, cuvettes, etc.). In embodiments of the invention, the term “container” refers to a well in a multiwell microplate or microtiter plate.

In embodiments of the invention, molecules that bind to the target molecule can be screened for their ability to bind to a target molecule in the presence of one or more co-regulators. The term “screening” refers generally to the testing of molecules or compounds for their ability to bind to a target molecule which is capable of denaturing or unfolding. The screening process can be a repetitive, or iterative, process, in which molecules are tested for binding to a protein in an unfolding assay.

As mentioned above, in accordance with embodiments of the invention, agonists or antagonists of a target molecule can be identified based upon modification of stability of the target molecule. Molecules that modify the stability of the target molecule can be screened for their ability to further modify the stability of the target molecule in the presence of one or more co-regulators.

In an embodiment, to perform the screening, one or molecules (e.g. of a set) that modify the stability of the target molecule can be contacted with the target molecule and one of more co-regulators in each of a multiplicity of containers. The target molecule in each of the containers can then be treated to cause the target protein to unfold. A physical change associated with the unfolding of the target molecule can be measured. An unfolding curve for the target molecule for each of containers can then be generated. Each of the unfolding curves may be compared to (1) each of the other unfolding curves and/or to (2) the unfolding curve for the target molecule in the absence of (i) any of the molecules from the set and/or (ii) the co-regulators.

Based upon the generated data, one can determine whether the screened molecules further modify the stability of the target molecule in the presence of the co-regulators, and thus identify whether the molecules are agonists or antagonists of the target molecule when the presence of the co-regulators. A further modification of stability of the target molecule is indicated by a further change in the unfolding curve of the target molecule.

In other embodiments of the invention, an agonist or antagonist of a co-regulator-dependent target molecule can be identified by an analysis of molecules that modify the thermal stability, and more particularly, shift the thermal unfolding curve of the target molecule. Molecules that shift the thermal unfolding curve of a target molecule can be screened for their ability to further shift the thermal unfolding curve of the target molecule in the presence of one or more co-regulators.

In an embodiment of the invention, the screening can be accomplished by contacting the target molecule with one or more of molecules (e.g., of a set) that shift the thermal unfolding curve of the target molecule with one or more co-regulators in each of a multiplicity of containers. The multiplicity of containers can be heated, and a physical change associated with the thermal unfolding curve for the target molecule as a function of temperature can be measured for each of the containers. A thermal unfolding curve for the target molecule as a function of temperature can then be generated. The thermal unfolding curves that are generated can be compared with (1) each of the other thermal unfolding curves and/or to (2) the thermal unfolding curve for the target molecule in the absence of (i) any of the molecules from the set and/or (ii) the co-regulators.

In embodiments of the screening method, the containers can be heated in intervals, over a range of temperatures. The multiplicity of containers may be heated simultaneously. A physical change associated with the thermal unfolding of the target molecule can be measured after each heating interval. In an alternate embodiment of this method, the containers can be heated in a continuous fashion.

In embodiments of the invention, in generating an unfolding curve for the target molecule, a thermal unfolding curve can be plotted as a function of temperature for the target molecule in each of the containers.

In an embodiment of the invention, comparing the thermal unfolding curves can be accomplished by comparing the midpoint temperatures, Tₘ of each unfolding curve. The “midpoint temperature, Tₘ” is the temperature midpoint of a thermal unfolding curve. The Tₘ can be readily determined using methods well known to those skilled in the art. See, for example, Weber, P. C. et al., 3. Am. Chem. Soc. 116:2717-2724 (1994); and Clegg, R. M. et al., Proc. Natl. Acad. Sci. U.S.A. 90:2994-2998 (1993).

For example, the Tₘ of each thermal unfolding curve can be identified and compared to the Tₘ obtained for (1) the other thermal unfolding curves and/or to (2) the thermal unfolding curve for the target molecule in the absence of (i) any of the molecules from the set and/or (ii) the co-regulators in the containers.

Alternatively or additionally, an entire thermal unfolding curve can be similarly compared to other entire thermal unfolding curves using computer analytical tools. For example, each entire thermal unfolding curve can be compared to (1) the other thermal unfolding curves and/or to (2) the thermal unfolding curve for the target molecule in the absence of (i) any of the molecules from the set and/or (ii) the co-regulators in the containers.

Based upon the generated data, one can determine whether any of the screened molecules further shift the thermal unfolding curve of the target molecule in the presence of a co-regulator, and thus identify whether a molecule is an agonist or antagonist of the target molecule when in the presence of a co-regulator.

The methods of the present invention that involve determining whether molecules that shift and/or further shift the thermal unfolding curve of a target molecule are distinct from methods that do not involve determining whether molecules shift and/or further shift the thermal unfolding curve of a target molecule, such as assays of susceptibility to proteolysis, surface binding by protein, antibody binding by protein, molecular chaperone binding of protein, differential binding to immobilized ligand, and protein aggregation. Such assays are well-known to those of ordinary skill in the art. For example, see U.S. Pat. No. 5,585,277; and U.S. Pat. No. 5,679,582. These approaches disclosed in U.S. Pat. Nos. 5,585,277 and 5,679,582 involve comparing the extent of folding and/or unfolding of the protein in the presence and in the absence of a molecule being tested for binding. These approaches do not involve a determination of whether any of the molecules that bind to the target molecule shift the thermal unfolding curve of the target molecule.

As discussed above, molecules that modify the stability of the target molecule can be screened for the ability to further modify the stability of the target molecule in the presence of a co-regulator. For example, molecules that are known to modify the stability of the target molecules can be screened against a panel of identified co-regulators for the target molecule, including co-activators and/or co-repressors. For convenience, the molecules known to modify the stability of the target molecule are referred to as a “sef” of molecules.

If the stability of the target molecule is further modified in the presence of a molecule from the set and a co-activator of the target molecule as compared to the target molecule and the molecule from the set alone, then this is an indication that the molecule from the set is an agonist of the target molecule when in the presence of the co-activator.

If the stability of the target molecule is further modified in the presence of a molecule from the set and a co-repressor of the target molecule as compared to the target molecule and the molecule from the set alone, then this is an indication that the molecule from the set is an antagonist of the target molecule when in the presence of the co-repressor.

Similarly, molecules that shift the thermal unfolding curve of the target molecule can be screened for the ability to further shift the thermal unfolding curve of the target molecule in the presence of a co-regulator. For example, molecules that are known to shift the thermal unfolding curve of the target molecule can be screened against a panel of identified co-regulators for the target molecule, including co-activators and/or co-repressors. For convenience, the molecules that are known to shift the thermal unfolding curve of the target molecule are referred to as a “set” of molecules.

If the thermal unfolding curve of the target molecule is further shifted in the presence of a molecule from the set and a co-activator of the target molecule as compared to the target molecule and the molecule from the set alone, then this is an indication that the molecule from the set is an agonist of the target molecule when in the presence of the co-activator.

If the thermal unfolding curve of the target molecule is further shifted in the presence of a molecule from the set and a co-repressor of the target molecule as compared to the target molecule and the molecule from the set alone, then this is an indication that the molecule from the set is an antagonist of the target molecule when in the presence of the co-repressor.

The present invention also provides methods for identifying agonists or antagonists of a co-regulator-dependent target molecule based on the lack of further modification of stability and/or a lack of further shift in the unfolding curve of a target molecule.

By “lack of further modification of stability of the target molecule” or “no further modification of stability of the target molecule,” it is meant that there is either an insignificant further change or no further change in the stability of the target molecule in the presence of both a molecule from the set and a co-regulator (as compared to the target molecule and the molecule from the set).

By “lack of further shift in the thermal unfolding curve of the target molecule” or “no further shift in the thermal unfolding curve of the target molecule,” it is meant that there is either an insignificant further change or no further change in the shift of the thermal unfolding curve of the target molecule in the presence of a molecule from the set and of a co-regulator (as compared to the target molecule and the molecule from the set).

In embodiments of the invention, an antagonist of a co-regulator-dependent target molecule can be identified based on the lack of further modification of stability and/or lack of further shift in the thermal unfolding curve of a target molecule when in the presence of a co-activator. In other embodiments of the invention, an agonist of a co-regulator-dependent target molecule can be identified based on the lack of further modification of stability and/or lack of further shift in the thermal unfolding curve of a target molecule when in the presence of a co-repressor.

An antagonist of a co-regulator-dependent target molecule can be identified by screening one or more of a set of molecules that modify the stability of the target molecule for their ability to further modify the stability of the target molecule in the presence of one or more co-activators. Methods for screening the molecules from the set for their effect on further modifying the stability of the target molecule are described above. If there is no further modification of the stability of the target molecule in the presence of a molecule of the set and a co-activator, then this is an indication that such molecule of the set is an antagonist of the target molecule when in the presence of the co-activator.

An antagonist can also be identified by screening one or more of a set of molecules that shift the thermal unfolding curve of the target molecule for their ability to further shift the thermal unfolding curve of the target molecule in the presence of one or more co-activators. Methods for screening one or more molecules of the set for their ability to further shift the thermal unfolding curve of the the target molecule are described above. If there is no further shift in the thermal unfolding curve of the target molecule in the presence of a molecule of the set and a co-activator, then this is an indication that such molecule of the set is an antagonist of the target molecule when in the presence of the co-activator.

An agonist of a co-regulator-dependent target molecule can be identified by screening one or more of a set of molecules that modify the stability of the target molecule for their ability to further modify the stability of the target molecule in the presence of one or more co-repressors. Methods for screening the molecules from the set for their effect on further modifying the stability of the target molecule are described above. If there is no further modification of the stability of the target molecule in the presence of a molecule of the set and a co-repressor, then this is an indication that such molecule of the set is an agonist of the target molecule when in the presence of the co-repressor.

An agonist can also be identified by screening one or more of a set of molecules that shift the thermal unfolding curve of the target molecule for their ability to further shift the thermal unfolding curve of the target molecule in the presence of one or more co-repressors. Methods for screening one or more molecules of the set for their ability to further shift the thermal unfolding curve of the the target molecule are described above. If there is no further shift in the thermal unfolding curve of the target molecule in the presence of a molecule of the set and a co-repressor, then this is an indication that such molecule of the set is an agonist of the target molecule when in the presence of the co-repressor.

Methods have been described above for the identification of agonists and antagonists of a co-regulator-dependent target molecule based on providing molecules that are known to modify the stability and/or shift the thermal unfolding curve of the target molecule and screening such molecules for their ability to further modify the stability of and/or shift the thermal unfolding curve of the target molecule. The invention also encompasses methods for the providing of such molecules in conjunction with the identification of such molecules as agonists or antagonists of the target molecule when in the presence of a co-regulator. Such methods are particularly useful in identifying ligands for orphan receptors, for which ligands that bind to the receptor are not known.

Molecules that modify the stability and/or shift the thermal unfolding curve of the target molecule (referred to above as a “set” for convenience) can be obtained by the screening of a multiplicity of different molecules. For example, molecules that modify the stability of the target molecule can be obtained by the screening of one or more of a multiplicity of different molecules for their ability to modify the stability of the target molecule. Similarly, molecules that shift the thermal unfolding curve of the target molecule can be obtained by the screening of one or more of a multiplicity of different molecules for their ability to shift the thermal unfolding curve of the target molecule. In embodiments of the invention, the number of molecules that can be screened range from about one thousand to one million.

Molecules can be screened for their ability to modify the stability of the target molecule by a method similar to the screening method described above for identifying agonists or antagonists. For example, the target molecule can be contacted with one or more of a multiplicity of different molecules in each of a multiplicity of containers. The target molecule in each of the multiplicity of containers can be treated to cause it to unfold. A physical change associated with the unfolding of the target molecule can be measured. An unfolding curve for the target molecule for each of the containers can be generated. Each of these unfolding curves can be compared to (1) each of the other unfolding curves and/or to (2) the unfolding curve for the target molecule in the absence of any of the multiplicity of different molecules.

Based upon the generated data, one can determine whether any of the screened molecules modify the stability of the target molecule. A modification of stability of the target molecule is indicated by a change in the unfolding curve of the target molecule. If a molecule modifies the stability of the target molecule, it can then be screened to identify whether it is an agonist or an antagonist of the target molecule when in the presence of a co-regulator by the methods described above.

Similarly, molecules can be screened for their ability to shift the thermal unfolding curve of the target molecule by a method similar to the screening method for identifying agonists or antagonists. For example, the target molecule can be contacted with one or more of a multiplicity of different molecules in each of a multiplicity of containers. The containers can be heated, and a physical change associated with the thermal unfolding of the target molecule can be measured in each of the containers. A thermal unfolding curve for the target molecule can be generated as a function of temperature for each of the containers.

The thermal unfolding curves can be compared with (1) each of the other thermal unfolding curves and/or to (2) the thermal unfolding curves for the target molecule in the absence of any of the multiplicity of different molecules. In embodiments of the invention, the Tₘ of each thermal unfolding curve can be identified and compared to the Tₘ obtained for (1) the other thermal unfolding curves and/or to (2) the thermal unfolding curve for the target molecule in the absence of any of the multiplicity of molecules. Alternatively, each entire thermal unfolding curve can be compared to (1) the other thermal unfolding curves and/or to (2) the thermal unfolding curve for the target molecule in the absence of any of the multiplicity of different molecules.

Based upon the generated data, one can determine whether any of the screened molecules shift the thermal unfolding curve of the target molecule. If a molecule shifts the thermal unfolding curve of the target molecule, it can then be screened to identify whether it is an agonist or an antagonist of the target molecule when in the presence of a co-regulator by the methods described above.

As discussed above, the methods of the present invention are particularly useful in identifying ligands for orphan receptors, for which ligands that bind to the receptor are not known. Similarly, the invention provides for a methods for identifying agonists and antagonists of a target molecule having an unknown function.

In an embodiment of the invention, a set of molecules is provided that modify the stability of a target molecule having an unknown function. This set of molecules modifies the stability of receptors which share biological function. The set of molecules that modify the stability of the target molecule can be provided by screening one or more panels of molecules which modify the stability of receptors which share biological function for their ability to modify the stability of the target molecule. Methods for providing such a set of molecules are described in more detail in U.S. Patent Publication No. U.S. 2001/0003648, herein incorporated by reference in its entirety.

One or more molecules of the set can be screened for their ability to further modify the stability of the target molecule in the presence of one or more co-regulators. As discussed in detail above, a further modification of the stability of the target molecule in the presence of a molecule of the set and a co-regulator indicates whether the molecule is an agonist or an antagonist of the target molecule when in the presence of the co-regulator. Embodiments of the invention include an identification of agonists and antagonists based upon no further modification of stability of the target molecule.

In another embodiment of the invention, a set of molecules are provided that shift the thermal unfolding curve of a target molecule having an unknown function. This set of molecules shifts the thermal unfolding curve of receptors which share biological function. The set of molecules that shift the thermal unfolding curve of the target molecule can be provided by screening one or more panels of molecules which shift the thermal unfolding curve of receptors which share biological function for their ability to modify the stability of the target molecule. Methods for providing such a set of molecules are also described in more detail in U.S. Patent Publication No. U.S. 2001/0003648.

One or more molecules of the set can be screened for their ability to further shift the thermal unfolding curve of the target molecule in the presence of one or more co-regulators. As discussed in detail above, a further shift in the thermal unfolding curve of the target molecule in the presence of a molecule of the set and a co-regulator indicates whether the molecule is an agonist or an antagonist of the target molecule when in the presence of the co-regulator. Embodiments of the invention also include an identification of agonists and antagonists based upon no further shift in the thermal unfolding curve of the target molecule.

In embodiments of the invention, a microplate thermal shift assay is a particularly useful means for identifying ligands and identifying such ligands as agonists or antagonists of co-regulator-dependent target molecules. The microplate thermal shift assay is a direct and quantitative technology for assaying the effect of one or more molecules on the thermal stability of a target receptor.

The theory, concepts, and application of the microplate thermal shift assay, and apparatuses useful for practicing the microplate thermal shift assay are described in U.S. Pat. Nos. 6,020,141; 6,036,920; 6,291,191; 6,268,218; 6,232,085; 6,268,158; 6,214,293; 6,291,192; and 6,303,322, which are all hereby incorporated by reference in their entireties. The microplate thermal shift assay discussed in these references can be used to implement the screening methods described above.

The microplate thermal shift assay provides a thermodynamic readout of ligand binding affinity. The assay depends upon the fact that each functionally active target molecule is a highly organized structure that melts cooperatively at a temperature that is characteristic for each target molecule and representative of its stabilization energy. When a molecule binds to a target molecule, the target molecule is stabilized by an amount proportional to the ligand binding affinity, thus shifting the midpoint temperature to a higher temperature.

There are many advantages to using the thermal shift assay since it does not require radioactively labeled compounds, nor fluorescent or other chromophobic labels to assist in monitoring binding. The assay takes advantage of thermal unfolding of biomolecules, a general physical chemical process intrinsic to many, if not all, drug target biomolecules. General applicability is an important aspect of this assay, as it obviates the necessity to invent a new assay every time a new therapeutic receptor protein becomes available.

Further, using the thermal shift assay, owing to the proportionality of the Tₘ and the ligand binding affinity, ligand binding affinities ranging from greater than 10 micromolar to less than 1 nanomolar can be measured in a single well experiment. Thus, the thermal shift assay can be used to quantitatively detect ligand binding affinity to a target molecule alone and/or in the presence of a co-regulator.

Further, the thermal shift assay can be used in the identification of agonists and antagonists on a quantitative basis based upon the change in the Tₘ between the ligand and target molecule and the ligand, target molecule and a co-regulator. The microplate thermal shift assay can be used to measure multiple ligand binding events on a single target molecule as incremental or additive increases of the target molecule's melting temperature.

The present invention has particular utility in the identification of ligands and the identification of such ligands as agonist or antagonist in nuclear receptors. For example, the present invention may be used to determine binding affinities for nuclear receptor ligands to predict in vivo efficacy, to discriminate ligands as agonist or antagonist to predict biological response, and to identify ligands for orphan receptors to discover their biological function.

For example, the present invention may be used to identify ligands that interact with the ligand binding domain of ER-α and ER-β, the two subtypes of the estrogen receptor family. These domains contain two known binding sites, one for estrogen like compounds and another for co-regulator proteins. The present invention can be used to identify ligands that interact with the estrogen receptor. These ligands produce an observed increase in the stability of the receptor which is proportional to the inherent affinity of the ligand.

The ligand binding domain of nuclear receptors, and co-regulator proteins can be expressed using standard recombinant methods in Escherichia coli. Co-regulator peptides can be synthesized using standard methods. The melting temperature of the purified protein of interest can be determined by the microplate thermal shift assay in the absence and in the presence of small molecule ligands.

Molecules are provided that stabilize the target molecule of interest. Such small molecules can be obtained by screening in the microplate thermal shift assay, as referred to above. The number of small molecules in the screen can range from about one thousand to one million. The small molecules can be natural or synthetic.

Once a set of small molecules have been identified to stabilize the protein of interest, then these molecules can be screened against a panel of co-regulators, such as proteins or peptide fragments, to measure their effect on the thermal stability of the protein. If a synergistic effect is observed, the compounds can be classified as agonist or antagonist. Equilibrium constants are calculated for both ligand and co-regulator and related to biological responses.

For assigning biological function to orphan receptors, the rate limiting step is the generation of a tool compound. One can screen the receptor of interest against a panel of compounds and identify ligands that stabilize the receptor of interest by the methods described above. Once ligands are identified, then one can screen against co-regulators to determine if the identified ligand is an agonist or an antagonist the methods described above.

Cell lines that contain the receptor of interest, as determined by, e.g., Western blot analysis, can be treated with the identified ligand. The ligand treated cell line can then be profiled for gene expression with DNA chips and compared against untreated cell lines. If the identified ligand is an agonist, a number of genes would be expected to be down-regulated when compared against the untreated cell line. Once this information is generated, the biological function of the receptor can be defined. This information, with the combination of chemi-informatics and bio-informatics can also assist in developing therapeutic hypothesis and testing them for the treatment of disease (see, e.g., Giguere, Endocrine Reviews 20:689-725 (1999), incorporated by reference herein in its entirety.)

Although the ligand binding domain of nuclear receptors, ligands and co-regulators that interact with this domain is described, the invention can be extended to the full length protein, in the presence of additional regulators and finally in the presence of DNA.

Further, it must be emphasized that the methods and the thermodynamic principles for data analysis can be used for any protein-protein interaction whose affinity is modulated by ligands or allosteric regulators. Examples can be and are not limited to GPCR's interacting with G-proteins to discriminate agonist from antagonist ligands; discriminating compounds that antagonize the association of SH2 domains to phophorylated forms of protein tyrosine kinases; identifying compounds that agonize or antagonize the PKA holoenzyme by affecting the oligomeric state of the enzyme; discriminating compounds that promote or inhibit the association of NF-KB to IKB; or compounds that promote or inhibit the oligomerization of transcription factors.

Also, these studies are not limited for protein-protein interactions but also can be used for protein-peptide interactions where the peptides represent short linear sequences representing protein domains that interact preferentially with the protein of interest.

Having now generally described the invention, the same will become more readily understood by reference to the following specific examples which are included herein for purposes of illustration only and are not intended to be limiting unless otherwise specified.

Experimental Results For Nuclear Receptors

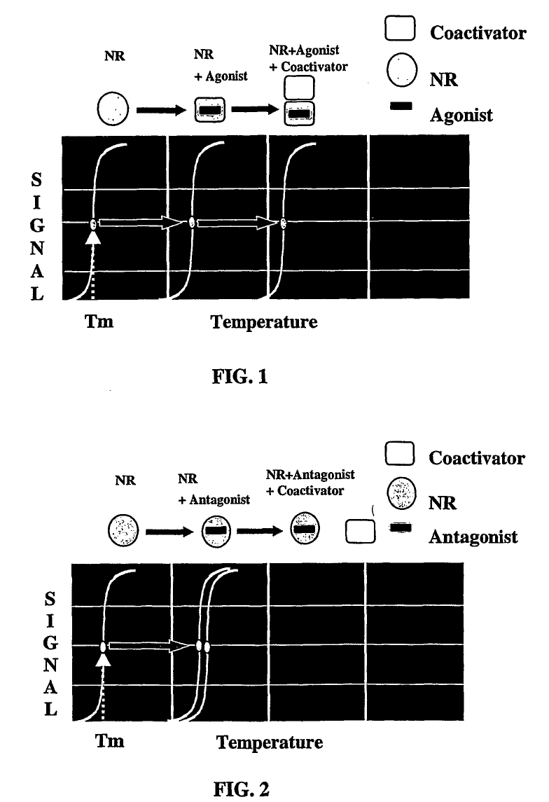

The experimental results expected for an agonist response vs. an antagonist response in the presence of a co-activator is shown in FIGS. 1 and 2. In the case of an agonist ligand and in the presence of co-activator protein/peptide the prediction is an increase in the stability of the receptor (FIG. 1), while for an antagonist no additional stabilization will be observed (FIG. 2).

### EXAMPLE 1

Table 1 is a summary of the data obtained for ER-c and ER-β for the study of a panel of four known agonist and three known antagonists in the presence of a co-activator protein SRC-3; in the presence of two co-activator peptides SRC1—NR2 and SRC3—NR2 derived from the sequence of the co-activators SRC-1 and SRC-3; and in the presence of the co-repressor peptide NCoR-1 derived from the co-repressor NCoR-1.

The concentration of ER-α and ER-β in all of the experiments was 8 μM, the ligand concentration was 20 μM, SRC-3 was 11 μM, and the co-regulator peptides SRC1—NR2, SRC3—NR2, and NCoR-1 was at 100 XM. The experiments were performed in 25 mM phosphate pH 8.0, 200 mM NaCl, 10% glycerol and in the presence of 25 μM dapoxyl sulfonamide dye (available from Molecular Probes, Inc., Eugene, Oreg.).

A 2 μL ligand solution at 2 times the final concentration was dispensed with a micropipette into a 384 well black wall Greiner plate. Then, 2 μL of the protein dye solution was dispensed on top of the ligand solution in the 384 well plate. The plates were spun to ensure mixing of the protein-dye and ligand solutions followed by layering of 1 μL of silicone oil to prevent evaporation during heating of the samples. Data were collected on a Thermofluor apparatus (see U.S. Pat. Nos. 6,020,141; 6,036,920; 6,291,191; 6,268,218; 6,232,085; 6,268,158; 6,214,293; 6,291,192; and 6,303,322) and analyzed using non-linear least squares fitting software. The results listed below are the average of four experiments. The values for the co-regulators represent a change in Tₘ stabilization from the receptor-ligand ΔTₘ values.

TABLE 1
Observed ΔTm stabilization of estrogen receptors
in the presence of ligands and coregulators.

| | — | SRC-3 | SRC-1 NR2 | SRC-3 NR2 | NCoR1 |
|---|---|---|---|---|---|
| ER-α | 0.0 | 0.5 | 0.8 | 0.9 | 0.0 |
| Estradiol | 13.4 | 3.8 | 4.9 | 4.3 | −0.2 |
| Estrone | 9.1 | 2.0 | 3.0 | 2.3 | −0.3 |
| 17-α-ethylen-E2 | 14.8 | 3.7 | 4.5 | 3.9 | −0.2 |
| 2-methoxy-E2 | 1.8 | 4.1 | 5.5 | 4.3 | −0.3 |
| tamoxifen | 9.4 | 0.5 | −0.5 | 0.0 | 0.1 |
| 4-OH-tamoxifen | 16.7 | 0.2 | 0.2 | 0.7 | 0.1 |
| ICI-182780 | 13.8 | 0.0 | 0.2 | 0.2 | −0.6 |
| ER-β | 0.0 | 0.4 | 0.7 | 0.9 | −0.4 |
| Estradiol | 16.4 | 1.1 | 3.4 | 3.5 | 0.0 |
| Estrone | 8.9 | 0.8 | 3.8 | 3.7 | −0.3 |
| 17-a-ethylene | 15.0 | 1.6 | 2.6 | 2.6 | −0.1 |
| 2-methoxy-E2 | 1.5 | 2.2 | 4.4 | 4.4 | −0.7 |
| tamoxifen | 6.1 | 0.2 | 0.2 | 0.4 | 0.4 |
| 4-OH-tamoxifen | 14.7 | 0.4 | 0.2 | 0.8 | 0.3 |
| ICI-182780 | 13.6 | 0.6 | 0.4 | 0.6 | 0.3 |

From the above results, from counter-screening in the presence of co-activator protein/peptide in the presence of the estrogen-like compounds, an additional stabilization was observed for both receptors. Thus, these compounds act like agonists in agreement with literature. The tamoxifen and ICI compound are known antagonists and they have no ability to recruit co-activators. This is also in agreement with the literature.

Also, the co-activator SRC-3 is preferentially recruited by ER-(c vs. ER-β. Therefore, the prediction is that these estrogen like compounds have a higher biological response in cell lines that contain ER-α vs. ER-β in the presence of SRC-3.

Further, the estrogen receptor does not have ability to recruit co-repressor peptide, therefore from a biological point of view the prediction is that gene repression will occur in ligand dependent fashion.

### EXAMPLE 2

ER-α was screened against a panel of steroid-like ligands to verify the ability of the methods of the present invention to determine ligands, and the function (see U.S. Patent Publication No. U.S. 2001/0003648 A1), of ER-(X if this receptor was classified as an orphan. Ligands that are known to interact with ER-α are identified as producing an increase in the stability of the receptor (compounds that are underlined versus those which are not underlined).

The concentration of ER-α in all of the experiments was 8 μM and the ligand concentration was 20 μM. The experiments were performed in 25 mM phosphate pH 8.0, 200 nM NaCl, 10% glycerol and in the presence of 25 μM dapoxyl sulfonamide dye (available from Molecular Probes, Inc., Eugene, Oreg.).

A 2 μL ligand solution at 2 times the final concentration was dispensed with a micropipette into a 384 well black wall Greiner plate. Then, 2 μL of the protein dye solution was dispensed on top of the ligand solution in the 384 well plate. The plates were spun to ensure mixing of the protein-dye and ligand solutions followed by layering of 1, of silicone oil to prevent evaporation during heating of the samples. Data were collected on a Thermofluor apparatus (see U.S. Pat. Nos. 6,020,141; 6,036,920; 6,291,191; 6,268,218; 6,232,085; 6,268,158; 6,214,293; 6,291,192; and 6,303,322) and analyzed using non-linear least-squares fitting software. The results listed below are the average of four experiments.

TABLE 2
Summary of data for ER-α in the presence of a panel of steroid ligands

| Steroid Ligand | ΔTm | Receptor target |
|---|---|---|
| 4-androstene | −0.23 | androgen receptor |
| androsterone | 0.23 | androgen receptor |
| corticosterone | −0.27 | glucocorticoid receptor |
| cortisone | 0.01 | glucocorticoid receptor |
| β-estradiol | 15.19 | estrogen receptor |
| estrone | 9.91 | estrogen receptor |
| 17-α-ethyleneestradiol | 18.72 | estrogen receptor |
| 17-α-hydroxyprogesterone | −0.21 | progesterone receptor |
| 2-methoxyestradiol | 5.98 | estrogen receptor |
| quabain | −0.21 | progesterone receptor |
| progesterone | −0.19 | progesterone receptor |
| 4-hydroxytamoxifen | 19.99 | estrogen receptor |

If ER-α was an orphan receptor, the data would had been interpreted that this receptor is a member of the estrogen receptor family. If the identified ligands that bind to the receptor had been screened against a panel of co-regulators, as in Example 1, β-estradiol, estrone, 17-α-ethyleneestradiol, and 2-methoxyestradiol are agonists for this receptor, while 4-hydroxytamoxifen is an antagonist. This data set demonstrates the utility of the microplate thermal shift assay for the identification of ligands for orphan receptors.

### EXAMPLE 3

Examples of other protein-protein interactions that may be analyzed using the present invention are illustrated in Table 2.

TABLE 2
Embodiment examples

| | Protein | | |
|---|---|---|---|
| Protein of | Partner (co- | Ligand | |
| Interest | regulator) | Phenotype | Related Biological Activity |
| GPCR | Gsα | Agonist | Increase cAMP or stimulate |
| | | | regulation of Ca²⁺ channels |
| GPCR | Giα | Agonist | Decrease cAMP |
| GPCR | Goα | Agonist | Stimulate regulation of Ca²⁺ |
| | | | channels |
| GPCR | Gtα | Agonist | Increase cGMP and |
| | | | phosphodiesterase activity |
| GPCR | Gqα | Agonist | Increase phospholipase Cβ |
| | | | activity |
| GPCR | Gsα | Antagonist | No effect on basal activity, or |
| | | | decrease cAMP, or inhibition of |
| | | | Ca²⁺ channel stimulation |
| GPCR | Giα | Antagonist | No effect on basal activity, or |
| | | | increase cAMP |
| GPCR | Goα | Antagonist | No effect on basal activity, or |
| | | | inhibition of Ca²⁺ channel |
| | | | stimulation |
| GPCR | Gtα | Antagonist | No effect on basal activity, or |
| | | | decrease cGMP and |
| | | | phosphodiesterase activity |
| GPCR | Gqα | Antagonist | No effect on basal activity, or |
| | | | decrease phospholipase Cβ |
| | | | activity |
| Src | SH2 | Antagonist | Inhibition of osteoclast mediated |
| | | | resorption of bone |
| Src | SH2 | Agonist | Stimulation of osteoclast |
| | | | mediated resorption of bone |
| Jac | SOCS | Agonist | Gene transcription |
| Jac | SOCS | Antagonist | Gene repression |
| NF-κB | IκB | Antagonist | Gene transcription |
| NF-κB | IκB | Agonist | Gene repression |

Different embodiments of this invention can include and are not limited to the examples above. The general nature of the examples contain the protein of interest, the interacting protein or peptide partner (co-regulator, e.g., a co-activator or co-repressor), and the ligand that can enhance (an agonist) or inhibit (an antagonist) the interaction.

While the foregoing invention has been described in some detail for the purposes of clarity and understanding, it will be appreciated by one skilled in the art from a reading of this disclosure that various changes in form and detail can be made without departing from the true scope of the invention and appended claims.

1-7. (canceled)

8. A method of identifying an agonist or an antagonist of a co-regulatordependent target molecule comprising providing a set of molecules that shift the thermal unfolding curve of the target molecule and screening one or more of said molecules of said set for their ability to further shift the thermal unfolding curve of the target molecule in the presence of one or more coregulators; wherein a further shift in the thermal unfolding curve of the target molecule in the presence of a molecule of said set and a co-regulator of said one or more co-regulators indicates whether the molecule is an agonist or an antagonist of the target molecule when in the presence of said co-regulator.

9. The method of claim 8, wherein providing the set of molecules that shift the thermal unfolding curve of the target molecule comprises screening one or more of a multiplicity of different molecules for their ability to shift the thermal unfolding curve of the target molecule.

10. The method of claim 9, wherein the screening of said one or molecules of said set of molecules for their ability to further shift the thermal unfolding curve of the target molecule comprises:
(a) contacting said target molecule and one or more molecules of said set with one or more of said co-regulators in each of a multiplicity of containers;
(b) heating said multiplicity of containers;
(c) measuring in each of said containers a physical change associated with the thermal unfolding of said target molecule resulting from said heating;
(d) generating a thermal unfolding curve for said target molecule as a function of temperature for each of said containers;
(e) comparing each of said thermal unfolding curves in step (d) to (1) each of the other thermal unfolding curves and/or to (2) the thermal unfolding curve for said target molecule in the absence of (i) any of said molecules of said set and/or (ii) said co-regulators, and
(f) determining whether any of said molecules of said set further shift the thermal unfolding curve of said target molecule.

11. The method of claim 10, wherein the screening of said one or more of a multiplicity of different molecules for their ability to shift the thermal unfolding curve of the target molecule comprises:
(a) contacting said target molecule with one or more of said multiplicity of different molecules in each of a multiplicity of containers;
(b) heating said multiplicity of containers;
(c) measuring in each of said containers a physical change associated with the thermal unfolding of said target molecule resulting from said heating;
(d) generating a thermal unfolding curve for said target molecules as a function of temperature for each of said containers;
(e) comparing each of said thermal unfolding curves in step (d) to (1) each of the other thermal unfolding curves and/or to (2) the thermal unfolding curve for said target molecule in the absence of any of said multiplicity of different molecules; and determining whether and of said multiplicity of different molecules shift the thermal unfolding curve of said target molecule.

12. The method of claim 8, wherein said one or more co-regulators includes a co-activator and/or a co-repressor.

13. The method of claim 12, wherein one or more molecules of the set further shift the thermal unfolding curve of the target molecule in the presence of a co-activator, thereby identifying an agonist of the target molecule when in the presence of the co-activator.

14. The method of claim 12, wherein the one or more molecules of the set further shift the thermal unfolding curve of the target molecule in the presence of a co-repressor, thereby identifying an antagonist of the target molecule when in the presence of the co-repressor.

15-18. (canceled)

19. A method of identifying an antagonist of a co-regulator-dependent target molecule comprising providing a set of molecules that shift the thermal unfolding curve of the target molecule and screening one or more of said molecules of said set for their ability to further shift the thermal unfolding curve of the target molecule in the presence of one or more co-activators; wherein no further shift in the thermal unfolding curve in the presence of a molecule of said set and a co-activator of said one or more-co-activators indicates that the molecule of said set is an antagonist of the target molecule when in the presence of said co-activator.

20. The method of claim 19, wherein providing the set of molecules that shift the thermal unfolding curve of the target molecule comprises screening one or more of a multiplicity of different molecules for their ability to shift the thermal unfolding curve of the target molecule.

21. The method of claim 20, wherein the screening of said one or molecules of said set of molecules for their ability to further shift the thermal unfolding curve of the target molecule comprises:
(a) contacting said target molecule and one or more molecules of said set with one or more of said co-activators in each of a multiplicity of containers;
(b) heating said multiplicity of containers;
(c) measuring in each of said containers a physical change associated with the thermal unfolding of said target molecule resulting from said heating;
(d) generating a thermal unfolding curve for said target molecule as a function of temperature for each of said containers;
(e) comparing each of said thermal unfolding curves in step (d) to (1) each of the other thermal unfolding curves and/or to (2) the thermal unfolding curve for said target molecule in the absence of (i) any of said molecules of said set and/or (ii) said co-activators; and
(f) determining whether any of said molecules of the set further shift the thermal unfolding curve of said target molecule.

22. The method of claim 21, wherein the screening of said one or more of a multiplicity of different molecules for their ability to shift the thermal unfolding curve of the target molecule comprises:
(a) contacting said target molecule with one or more of said multiplicity of different molecules in each of a multiplicity of containers;
(b) heating said multiplicity of containers;
(c) measuring in each of said containers a physical change associated with the thermal unfolding of said target molecule resulting from said heating;
(d) generating a thermal unfolding curve for said target molecule as a function of temperature for each of said containers;
(e) comparing each of said thermal unfolding curves in step (d) to (1) each of the other thermal unfolding curves and/or to (2) the thermal unfolding curve for said target molecule in the absence of any of said multiplicity of different molecules; and
(f) determining whether any of said multiplicity of different molecules shift the thermal unfolding curve of said target molecule.

23-26. (canceled)

27. A method of identifying an agonist of a co-regulator-dependent target molecule comprising providing a set of molecules that shift the thermal unfolding curve of the target molecule and screening one or more of said molecules of said set for their ability to further shift the thermal unfolding curve of the target molecule in the presence of one or more co-repressors; wherein no further shift in the thermal unfolding curve in the presence of a molecule of said set and a co-repressor of said one of more-co-repressors indicates that the molecule of said set is an agonist of the target molecule when in the presence of said co-repressor.

28. The method of claim 27, wherein providing the set of molecules that shift the thermal unfolding curve of the target molecule comprises screening one or more of a multiplicity of different molecules for their ability to shift the thermal unfolding curve of the target molecule.

29. The method of claim 28, wherein the screening of said one or molecules of said set of molecules for their ability to further shift the thermal unfolding curve of the target molecule comprises:
(a) contacting said target molecule and one or more molecules of said set with one or more of said co-repressors in each of a multiplicity of containers;
(b) heating said multiplicity of containers;
(c) measuring in each of said containers a physical change associated with the thermal unfolding of said target molecule resulting from said heating;
(d) generating a thermal unfolding curve for said target molecule as a function of temperature for each of said containers;
(e) comparing each of said thermal unfolding curves in step (d) to (1) each of the other thermal unfolding curves and/or to (2) the thermal unfolding curve for said target molecule in the absence of (i) any of said molecules of said set and/or (ii) said co-repressors; and
(f) determining whether any of said molecules of the set further shift the thermal unfolding curve of said target molecule.

30. The method of claim 29, wherein the screening of said one or more of a multiplicity of different molecules for their ability to shift the thermal unfolding curve of the target molecule comprising:
(a) contacting said target molecule with one or more of said multiplicity of different molecules in each of a multiplicity of containers;
(b) heating said multiplicity of containers;
(c) measuring in each of said containers a physical change associated with the thermal unfolding of said target molecule resulting from said heating;
(d) generating a thermal unfolding curve for said target molecule as a function of temperature for each of said containers;
(e) comparing each of said thermal unfolding curves in step (d) to (1) each of the other thermal unfolding curves and/or to (2) the thermal unfolding curve for said target molecule in the absence of any of said multiplicity of different molecules; and
(f) determining whether any of said multiplicity of different molecules shift the thermal unfolding curve of said target molecule.

31-34. (canceled)

35. The method of claim 8, wherein the target molecule is a nuclear receptor.

36. The method of claim 8, wherein the target molecule is a G-protein coupled receptor.

37. The method of claim 8, wherein the target molecule is ER-α.

38. The method of claim 8, wherein the target molecule is ER-β.

39. The method of claim 8, wherein the target molecule is a tyrosine kinase.

40. The method of claim 8, wherein the target molecule is a NF-κB.
